(19) United States
(12) Patent Application Publication
(10) Pub. No.: US 2019/0120828 A1
(43) Pub. Date: Apr. 25, 2019

(54) PREGNANCY TEST AND KEEPSAKE DEVICE AND METHOD OF USE THEREOF

(71) Applicants: Angela Renee Crumitie, Ocoee, FL (US); Erica Mechelle Thompson, Ocoee, FL (US)

(72) Inventors: Angela Renee Crumitie, Ocoee, FL (US); Erica Mechelle Thompson, Ocoee, FL (US)

(21) Appl. No.: 16/222,427

(22) Filed: Dec. 17, 2018

Related U.S. Application Data
(63) Continuation-in-part of application No. 15/336,621, filed on Oct. 27, 2016.

(51) Int. Cl. G01N 33/53; G01N 33/76

(52) U.S. Cl. CPC G01N 33/5302; G01N 2333/59; G01N 33/76

(57) ABSTRACT

The embodiments disclosed provide an at-home pregnancy test device comprising a test strip retained within an elongated housing configured in the shape of a human. The elongated housing has an opening at an end configured to receive a removable sample collector adapted to receive a sample from a user. The elongated housing is configured to stand on a flat lower surface as a keepsake.

### CROSS-REFERENCE TO RELATED APPLICATIONS

This application is a continuation-in-part of non-provisional patent application Ser. No. 15/336,621 filed on Oct. 27, 2016, entitled “The Curve of Life Pregnancy Test” by the present inventors.

### FIELD

The present embodiment relates to pregnancy tests and more specifically relates to pregnancy tests which are configured as a keepsake.

### BACKGROUND

Pregnancy tests offer at at-home means for determining whether or not a woman is pregnant by using indicative biological markers present in the blood or urine. Most tests analyze urine from the female for the beta subunit of human chorionic gonadotropin (hCG) which is only produced six to twelve days following fertilization. Other rapid tests include the rosette inhibition assay for early pregnancy factor (EPF) which can be detected within 48 hours of fertilization.

Modern pregnancy tests provided by pharmacies and similar retailers include an immunoassay strip at least partially contained in a housing. The housing is often a simple plastic component with a viewing area wherein test results are displayed. During the production of the pregnancy test device, the immunoassay strip is coated with a monoclonal antibody which will react with any hCG present in the urine. The reaction between the monoclonal antibody and hCG results in the generation of a control line if the test is viable, as well as positive and negative lines. Following the reception of the test results, the pregnancy test device is often discarded, as the components are intended to be single-use.

### SUMMARY OF THE INVENTION

The embodiments disclosed provide an at-home pregnancy test device comprising a test strip retained within an elongated housing configured in the shape of a human. The elongated housing has an opening at an end configured to receive a removable sample collector adapted to receive a sample from a user. The elongated housing is configured to stand on a flat lower surface as a keepsake.

In one aspect, the sample collector is provided at a bottom end. The elongated housing is comprised of a central portion extending between a top portion and the bottom end.

In one aspect, the central portion is comprised of a viewing window to facilitate viewing of a test result provided on the test strip.

In one aspect, the top portion is comprised of a hair region configured as a button such that the button facilitates the removal of the sample collector.

In one aspect, the test strip is coated with a monoclonal antibody configured to react with a component of the sample. The component can include hCG, wherein the presence of hCG results in a positive test result.

In one aspect, a user performing the steps of performing a pregnancy test by providing the sample to the sample collector. The user then determines the result of the pregnancy test. If the test result is positive, the user removes the sample collector from the housing and retains the housing as a keepsake. To facilitate the retainment of the keepsake, the housing may include a flat surface providing a suitable standing surface.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates a front elevation view of the pregnancy test device, according to some embodiments;

FIG. 2 illustrates a rear elevation view of the pregnancy test device, according to some embodiments;

FIG. 3 illustrates a side elevation view of the pregnancy test device, according to some embodiments; and

FIG. 4 illustrates a flowchart of the method of use for the pregnancy test device, according to some embodiments.

### DETAILED DESCRIPTION

The specific details of the single embodiment or variety of embodiments described herein are to the described device and methods of use. Any specific details of the embodiments are used for demonstration purposes only and not unnecessary limitations or inferences are to be understood therefrom.

Before describing in detail exemplary embodiments, it is noted that the embodiments reside primarily in combinations of components related to the system and method. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

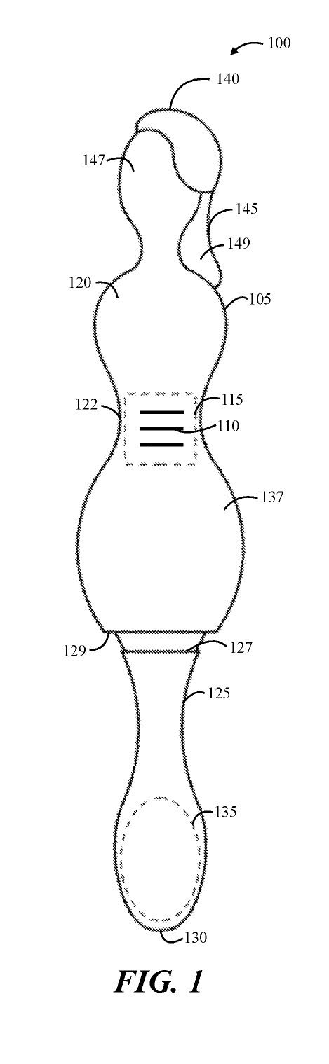

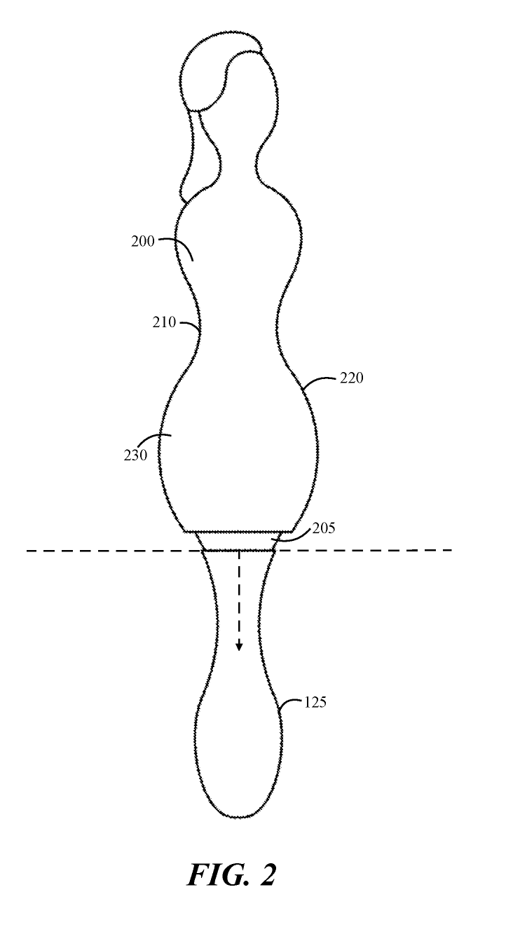

FIG. 1 and FIG. 2 illustrate the pregnancy test device 100 in exemplary embodiments. The device 100 is comprised of a housing 105 configured to have a distinct shape, such as that of a human. In some embodiments, the housing 105 is configured in the shape of a female. The housing retains a test strip 110 as known in the arts.

The test strip 110 can be provided using any known test for pregnancy, such as a test strip coated or treated with a reactive substance such as with monoclonal antibodies which bind to the beta subunit of human chorionic gonadotropin (hCG). If binding occurs between the hCG and the monoclonal antibodies on the test strip, a positive test result is displayed. One skilled in the arts will readily understand that a variety of biochemical pregnancy test methods may be employed.

An aperture 115 is positioned on the front side 120 of the housing 105 to allow the user to view the test results. Test results are typically displayed as one or more indicator lines. Indicator lines can include a control line, a negative line, and a positive line. One skilled in the arts will readily understand that various indicators may be used which can be displayed having one or more colors, shapes, or combinations to identify a valid test, a negative test result, or a positive test result. As shown in FIG. 1, the test result indicators are commonly provided as a series of lines. However, the indicator can include various shapes and colors to indicate valid, positive, and negative tests. For example, the indicator can be provided in the shape of a baby, an embryo, or other shapes.

The housing 105 is comprised of a central portion 122 which may be shaped like the torso of a human. A sample collector 125 extends between the lower surface 127 of the central portion 122 to the bottom end 130. The sample collector 125 includes a receptacle 135 configured to receive urine from the user and transfer the urine via capillary action, or other known means, to the test strip 110. The receptacle is provided on the front side 137 of the sample collector 125.

In some embodiments, the sample collector 125 is releasably engaged with the central portion 122 via retainer 205 (shown in FIG. 2). The retainer 205 is positioned between the central portion 122 and the sample collector 125 to facilitate the removal thereof following use. The flat surface 127 of the retainer 205, as well as the surface 129 of the of the central portion 122, permit the device 100 to be stood on an end as a keepsake following the removal of the sample collector 125.

The housing 105 can be provided as an elongated cavity wherein the test strip 110 is retained. An opening receives the retainer 205. The elongated housing 105 enables the use of an elongated sample collector 125, which, in combination of the further features makes this embodiment especially suited for home-use detection of pregnancy and retaining the device 100 as a keepsake.

The housing 105 is produced from a material which is impermeable to moisture, such as thermoplastic material, polystyrene or the like, and acts to receive and transfer a certain amount of the test liquid. For this purpose, the housing 105 is equipped with an interior space and an opening for collection of the sample and optionally means via which contact of the sample with the test strip can take place.

The test strip 110 consists of a material which transports the test liquid essentially by capillary forces such as absorbent, porous or fibrous material suitable for rapid uptake of liquid. Suitable materials may include materials such as polypropylene, polyethylene, polyvinylidene fluoride, ethylene/vinyl acetate copolymer, polyacrylonitrile, and polytetrafluoroethylene. Other suitable materials include multilayer materials and materials which are already generally used in analytical test strips such as paper or paper-like materials, for example, nitrocellulose.

The retainer 205 can releasably engage with the sample collector 125 and/or the central portion 122 by a friction fit, snap-fit mechanism, locking mechanism, or similar means.

The housing 105 can also include a top portion 140 extending between the central portion 122 and the top end 145. In some embodiments, the top portion 140 is configured as a head of a human having a facial region 147 and a hair region 149.

In some embodiments, the hair region 149 is configured as a button facilitating the removal of the sample collector 125 via the retainer 205.

The sample collector includes a front side 137, as shown in FIG. 1. FIG. 2 illustrates the sample collector rear side 230, in addition to left and right sides 210,220. The rear side 230 of the sample collector is on the same side as the back side 200 of the housing, while the front side 137 is on the same side as the housing 105 front side 120.

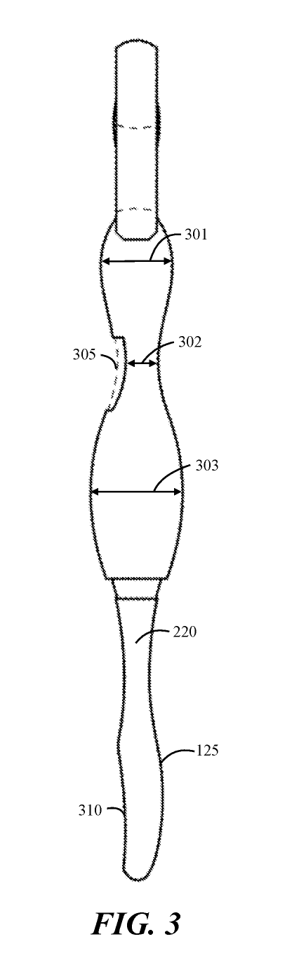

FIG. 3 illustrates the right side 220 of the sample collector 125 having a bowl portion 310 which facilitates the collection of the sample from the user. The central portion may include a top region having a first width 301, a middle region having a second width 302, and a lower region having a third width 303. Each of the first, second, and third widths (301,302,303 respectively) are dimensioned to correspond to the general shape of the human body. In one example, the third width 303 is the largest value, the second width 302 is the smallest value, and the first width 301 is the middle value.

In some embodiments, the aperture 115 is covered by a viewing window 305 (shown in FIG. 3) which protects the test strip 110 from tampering and degradation over time. The viewing window may protrude from the surface of the housing.

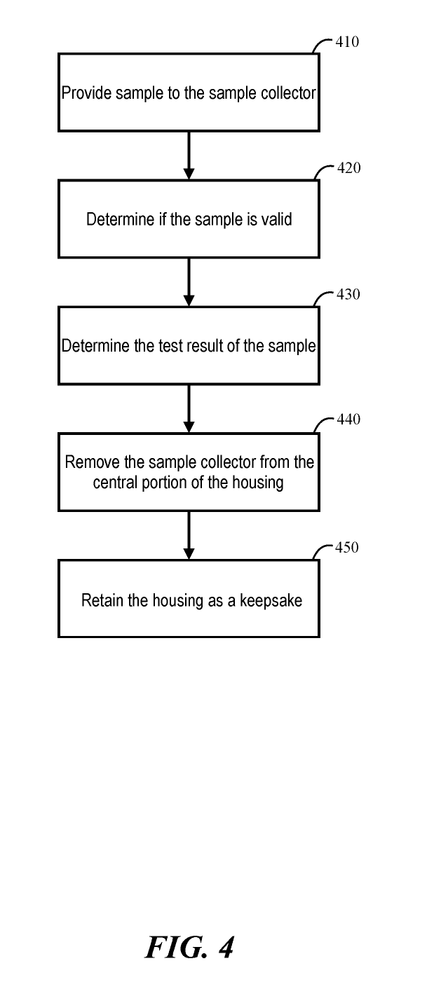

FIG. 4 illustrates a flowchart of a method of use for the pregnancy test and keepsake. In step 410, the user provides a sample to the sample collector and determines if the sample is valid in step 420 by viewing the indicators on the test strip. The viewing window may facilitate the viewing of the test strip and indicators. In step 430, the user determines the test result, which can be invalid, positive, or negative. If the test is positive, the user removes the sample collector from the housing in step 440 and retains the housing as a keepsake in step 450.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. An at-home pregnancy test device comprising:
a test strip retained within an elongated housing configured in the shape of a human, the elongated housing having an opening at an end configured to receive a removable sample collector adapted to receive a sample from a user, the elongated housing configured to stand on a flat lower surface as a keepsake.

2. The device of claim 1, wherein the sample collector is provided at a bottom end, wherein the elongated housing is comprised of a central portion extending between a top portion and the bottom end.

3. The device of claim 2, wherein the central portion is comprised of a viewing window to facilitate viewing of a test result provided on the test strip.

4. The device of claim 3, wherein the top portion is comprised of a hair region and configured as a button, wherein the button facilitates the removal of the sample collector.

5. The device of claim 1, wherein the test strip is coated with a monoclonal antibody configured to react with a component of the sample.

6. The device of claim 5, wherein the component is hCG, wherein the presence of hCG results in a positive test result.

7. A system for utilizing an at-home pregnancy test device as a keepsake, the system comprising:
a test strip retained within an elongated housing configured in the shape of a human, the elongated housing having an opening at an end configured to receive a removable sample collector adapted to receive a sample from a user, the elongated housing configured to stand on a flat lower surface as a keepsake;
a user performing the steps of:
performing a pregnancy test by providing the sample to the sample collector;
determining a result of the pregnancy test;
removing the sample collector from the housing; and
retaining the housing as the keepsake.

8. The system of claim 7, wherein the housing includes a flat surface providing a suitable standing surface for the keepsake.

9. The system of claim 7, wherein the sample collector is provided at a bottom end, wherein the elongated housing is comprised of a central portion extending between a top portion and the bottom end.

10. The system of claim 9, wherein the central portion is comprised of a viewing window to facilitate viewing of the test result provided on the test strip.

11. The system of claim 9, wherein the top portion is comprised of a hair region configured as a button, wherein the button facilitates the removal of the sample collector.

12. The system of claim 7, wherein the test strip is coated with a monoclonal antibody configured to react with a component of the sample.

13. The system of claim 12, wherein the component is hCG, wherein the presence of hCG results in a positive test result.

14. A system for utilizing an at-home pregnancy test device as a keepsake, the system comprising:
a test strip treated with a monoclonal antibody to hCG, the test strip retained within an elongated housing including a viewing window positioned at a central portion, the elongated housing having an opening at an end configured to receive a removable sample collector adapted to receive a sample from a user, the elongated housing configured to stand on a flat lower surface as a keepsake;
a retainer removably engaged with the sample collector, the retainer in operable communication with a button facilitating the removal of the sample collector via the retained, the button positioned on a hair portion of the housing;
a user performing the steps of:
performing a pregnancy test by providing the sample to the sample collector;
determining a result of the pregnancy test;
removing the sample collector from the housing; and
retaining the housing as the keepsake.

15. The system of claim 14, wherein the housing includes a flat surface providing a suitable standing surface for the keepsake.

16. The system of claim 14, wherein the sample collector is provided at a bottom end, wherein the elongated housing is comprised of a central portion extending between a top portion and the bottom end.

17. The system of claim 14, wherein the presence of hCG in the sample results in a positive test result, wherein an indicator indicating the positive result is maintained in viewable position through the viewing window.

18. The system of claim 17, wherein the viewing window is provided on a front side of the housing.

19. The system of claim 17, wherein the sample collector is further comprised of a bowl portion to facilitate the collection of the sample.

20. The system of claim 14, wherein the housing is constructed of an impermeable material.
